(19) United States
(12) Plant Patent Application Publication
(10) Pub. No.: US 2003/0188366 P1
(43) Pub. Date: Oct. 2, 2003

(54) PETUNIA PLANT NAMED 'SUNBEL-APU'

(76) Inventor: Yasuyuki Murakami, Gamo-gun (JP)

Correspondence Address: BURNS, DOANE, SWECKER & MATHIS, L.L.P., P.O Box 1404, Alexandria, VA, 22313-1404, US

(21) Appl. No.: 10/107,192

(22) Filed: Mar. 28, 2002

(51) Int. Cl. A01H 5/00

(52) U.S. Cl. PLT/356

(57) ABSTRACT

Disclosed herein is a new and distinct variety of Petunia plant having a semi-erect habit. The Petunia plant has abundant branching particularly with respect to secondary branching, and a great profusion of-blooms with the entire plant remaining in bloom for a considerable period of time. The flowers are very small with petals having a light yellowish pink color with vivid reddish orange center margin, and having a plentiful number of flower in a spike. The blooming period is from May to September and the flowering duration is long. The plant exhibits high resistance to cold, rain, heat, drought and diseases such as Powdery Mildew. The resistance to rain and heat is very strong.

### BACKGROUND OF THE VARIETY

The present invention relates to a new and distinct variety of petunia plant obtained from crossing of a petunia plant variety called ‘E2 and another petunia plant variety called ‘PE7’.

The petunia is a very popular plant and is used for flower bedding and potting in the summer season. There are only a few varieties of the Petunia plants which do not have an upright growth habit and which have a high resistance to rain, heat, cold and diseases. Recently, we have developed a series of small flower petunias, such as ‘SUNTORY SP-R’ (U.S. Plant Pat. No. 9557), ‘SUNBERUBU’ (U.S. Plant Pat. No. 9754), ‘SUNBELOCHIPI’ (U.S. Plant Pat. No. 10,355) and ‘SUNBELKI’ (U.S. Plant Pat. No. 11,558) which are of the semi-erect type, have short stems, a medium plant height, abundant branching, and a high resistance to heat, cold and rain. However, there are only a few varieties having a great profusion of flowers, a light yellowish pink with vivid reddish orange flower color, a very small flower size and a high resistance to rain, heat, cold and diseases. Accordingly, this invention was aimed at obtaining a new variety having a light yellowish pink with vivid reddish orange flower color, and very small flowers combined with the above features.

The new variety of Petunia plant according to this invention originated from crossing of a petunia plant variety called ‘E2’ and the other petunia plant variety called ‘PE7’.

First of all, 10 seedlings of petunia plant (PE1-PE10) were selected in view of yellowish pink petal color among the small type petunia plant that had been grown at Kitakoma-gun, Yamanashi-ken, Japan in 1994. From 1995 to 1996, open-pollination with those 10 seedlings was conducted at Youkaichi-shi, Shiga-ken, Japan. From that crossing, seedlings were obtained which are called ‘E-series’. Then, seedlings were obtained from crossing with PE1-PE10 and E-series. In 1997, some seedlings were selected among them in view of small flower size, light yellowish pink petal with vivid reddish orange margin, florifelous and semi-erect habit. These selected seedlings were grown in beds and pots on trial at Kitakoma-gun, Yamanashi-ken, Japan. Finally, in September 1998, one petunia plant was selected. In 1999, the botanically characteristics of these seedlings were examined, using similar varieties ‘SUNBELKI’ (registration No. 8962 under the Seeds and Seedlings Law of Japan) and ‘Pearl Sky Blue’ (unpatented under the Seeds and Seedlings Law of Japan) for comparison. As a result, it was concluded that this petunia is distinguishable from any other variety, whose existence is known to us, uniform and stable in its characteristics, and this new variety of petunia plant was named ‘SUNBEL-APU’.

In the following description, the color-coding is in accordance with the Horticultural Color Chart of The Royal Horticultural Society, London, England (R.H.S. Color Chart), Inter-Society Color Council—National Bureau of Standards (ISCC-NBS), and a color chart based on The Japan Color Standard for Horticultural Plant (J.H.S. Color Chart) has also been added for reference.

Type ‘E2’ used as the female parent in the obtaining of this new variety ‘SUNBEL APU’ is our bred variety and was not filed in Japan or other countries. Type ‘E2’ was obtained from open-pollination with the small type petunia plant that had been grown at Kitakoma-gun, Yamanashi-ken, Japan. The main botanical characteristics of ‘E2’ are as follows.

Plant:

Growth habit.—Semi-erect.

Plant height.—medium; 14 cm.

Spreading area of plant.—Medium to large; 18 cm.

Blooming period.—May to September in the southern Kanto area, Japan.

Stem:

Thickness.—Thin; 2.1 mm.

Pubescence.—Sparse.

Branching.—Abundant with a superior branching propensity. The secondary branches are particularly numerous.

Length of internode.—Short; 1.0 cm.

Leaf:

Shape.—Lanceolate.

Length.—Very short; 2.4 cm.

Width.—Very narrow; 1.1 cm.

Color.—Grayish olive green. (R.H.S. color chart No. 137B, JHS color chart No. 3716).

Thickness.—Thin; 0.3 mm.

Pubescence.—Sparse to normal.

Leaf attachment angle to stem.—Slanted upward to horizontal.

Flower:

Facing direction.—Slanted upward to horizontal.

Type.—Single.

Shape.—Funnel-shape, with five petals.

Shape of petal tip.—Round.

Waving of petal.—Weak.

Lobation of petal.—Shallow.

Diameter.—Very small; 2.7 cm.

Variegated pattern of corolla.—Margin.

Ground color of bi-colored petal.—Pale orange yellow (R.H.S. color chart No. 23D, JHS color chart No. 1903).

Color of center part.—Vivid reddish orange (R.H.S. color chart No. 34A, JHS color chart No. 0705).

Bottom color of the corolla throat.—Brilliant yellow (R.H.S. color chart No. 15B, JHS color chart No. 2204).

Outside color of corolla tube.—Brilliant yellow (R.H.S. color chart No. 13C, JHS color chart No. 2505).

Reproductive organs.—1 normal pistil and 5 normal stamens.

Fertility.—Fertile, but self-incompatible.

Peduncle.—0.6 mm in thickness, and 1.9 cm in length.

Physiological and ecological characteristics.—High resistance to cold, rain, heat, drought and diseases such as Powdery Mildew. The resistance to rain and heat is very strong.

Blooming.—A bloom commonly lasts approximately 10 days on the plant. Pinching is not necessary to ensure continued blooming; however, it does tend to enhance bloom production somewhat.

Type ‘PE7’ used as the male parent in obtaining of this new variety ‘SUNBEL-APU’ is our bred variety and was not filed in Japan or another country. Type ‘PE7’ was selected in view of yellowish pink petal color among the small type petunia plants that had been grown at Kitakoma-gun, Yamanashi-ken, Japan. The main botanical characteristics of ‘PE7’ are as follows.

Plant:

Growth habit.—Medium to spread.

Plant height.—Medium; 14 cm.

Spreading area of plant.—Medium; 16 cm.

Blooming period.—May to September in the southern Kanto area, Japan.

Stem:

Thickness.—Thin; 1.4 mm.

Pubescence.—Sparse.

Branching.—Abundant with a superior branching propensity. The secondary branches are particularly numerous.

Length of internode.—Short; 1.2 cm.

Leaf:

Shape.—Lanceolate.

Length.—Very short; 2.0 cm.

Width.—Very narrow; 0.7 cm.

Color.—Grayish olive green. (R.H.S. color chart No. 137A, JHS color chart No. 3716).

Thickness.—Thin; 0.4 mm.

Pubescence.—Sparse to normal.

Leaf attachment angle to stem.—Slanted upward to horizontal.

Flower:

Facing direction.—Slanted upward.

Type.—Single.

Shape.—Funnel-shape, with five petals.

Shape of petal tip.—Round.

Waving of petal.—Weak.

Lobation of petal.—Shallow.

Diameter.—Very small; 2.4 cm.

Variegated pattern of corolla.—Margin.

Ground color of bi-colored petal.—Strong yellowish pink (R.H.S. color chart No. 38A, JHS color chart No. 0704).

Color of variegated pattern.—Vivid red (R.H.S. color chart No. 42A, JHS color chart No. 0707).

Bottom color of the corolla throat.—Brilliant yellow (R.H.S. color chart No. 15A, JHS color chart No. 2204).

Outside color of corolla tube.—Vivid yellow (R.H.S. color chart No. 13A, JHS color chart No. 2506).

Reproductive organs.—1 normal pistil and 5 normal stamens.

Fertility.—Fertile, but self-incompatible.

Peduncle.—0.7 mm in thickness, and 1.2 cm in length.

Physiological and ecological characteristics.—High resistance to cold, rain, heat, drought and diseases such as Powdery Mildew.

Blooming.—A bloom commonly lasts approximately 10 days on the plant. Pinching is not necessary to ensure continued blooming; however, it does tend to enhance bloom production somewhat.

Type ‘SUNBELKI’ used as a comparison for this new variety ‘SUNBEL-APU’ is our bred variety and was registered in Japan in 2001 (registration No. 8962 under the Seeds and Seedlings Law of Japan) and in the U.S. in 2000 (U.S. Plant Pat. No. 11558), and so on. The main botanical characteristics of ‘SUNBELKI’ are as follows.

Plant:

Growth habit.—Decumbent to medium.

Plant height.—18 cm.

Spreading area of plant.—The stem extends to a length of 18 cm from the base.

Growth.—Very vigorous with abundant branching, and a great profusion of blooms, with the whole bush remaining in bloom for a considerable period of time.

Blooming period.—April to late October in the southern Kanto area, Japan. The plant shape does not change throughout this period.

Stem:

Thickness.—2.3 mm.

Pubescence.—Present.

Branching.—Abundant with a superior branching propensity. The secondary branches are particularly numerous.

Length of internode.—1.5 cm.

Leaf:

Shape.—Lanceolate.

Length.—5.0 cm.

Width.—1.4 cm.

Color.—Moderate olive green. (R.H.S. color chart No. 146B, JHS color chart No. 3508).

Thickness.—Very thin, 0.2-0.4 mm.

Pubescence.—Sparse.

Leaf attachment angle to stem.—Slanted upward to horizontal.

Flower:

Facing direction.—Horizontal.

Type.—Single.

Shape.—Funnel-shape, with five petals.

Shape of petal tip.—Mucronate.

Waving of petal.—Weak.

Lobation of petal.—Shallow.

Diameter.—2.8 cm.

Color.—Petal: Brilliant yellow (R.H.S. color chart No. 12A, JHS color chart No. 2505). At low temperatures, it only displays a dark reddish brown (R.H.S. color chart No. 166A, JHS color chart No. 1310) main vein and a grayish reddish brown (R.H.S. color chart No. 165A, JHS color chart No. 1317) secondary vein on the petals.

Bottom color of the corolla throat.—Strong brown (R.H.S. color chart No. 166B, JHS color chart No. 1308) with a slightly dark reddish brown (R.H.S. color chart No. 166A, JHS color chart No. 1310) veins.

Outside color of corolla tube.—Light greenish yellow (R.H.S. color chart No. 4C, JHS color chart No. 2904) with a slightly dark reddish brown (R.H.S. color chart No. 166A, JHS color chart No. 1310) veins.

Reproductive organs.—1 normal pistil and 5 normal stamens.

Fertility.—Seeds are formed, but are infertile from observations to date.

Peduncle.—Thin, 0.7-0.8 mm in thickness, and 1.9 cm in length.

Physiological and ecological characteristics.—High resistance to rain, heat, drought and diseases such as Powdery Mildew. The resistance to rain and heat is very strong.

Blooming.—A bloom commonly lasts approximately 10 days on the plant. Pinching is not necessary to ensure continued blooming; however, it does tend to enhance bloom production somewhat.

The botanical characteristics of the Petunia plant ‘Pearl Sky Blue’ which was used as a comparison for this new variety ‘SUNBEL-APU’ are as follows.

Plant:

Growth habit.—Medium to spread.

Plant height.—Medium; 14 cm.

Spreading area of plant.—Medium to large; 16 cm.

Blooming period.—May to September in the southern Kanto area, Japan.

Stem:

Thickness.—Thin; 2.7 mm.

Pubescence.—Dense.

Branching.—Abundant.

Length of internode.—Short; 1.3 cm.

Leaf:

Shape.—Elliptic.

Length.—Short; 5.5 cm.

Width.—Narrow to medium; 3.5 cm.

Color.—Strong yellow green. (R.H.S. color chart No. 144A, JHS color chart No. 3507).

Thickness.—Medium.

Pubescence.—Dense.

Leaf attachment angle to stem.—Horizontal to droopy.

Flower:

Facing direction.—Upward.

Type.—Single.

Shape.—Funnel-shape, with five petals.

Shape of petal tip.—Round.

Waving of petal.—Weak.

Lobation of petal.—Shallow.

Diameter.—Small; 5.5 cm.

Variegated pattern of corolla.—Single color.

Color.—Petal: Strong purple (R.H.S. color chart No. 83C, JHS color chart No. 8310).

Bottom color of the corolla throat.—Light greenish yellow (R.H.S. color chart No. 3D, JHS color chart No. 2904).

Outside color of corolla tube.—Light greenish yellow (R.H.S. color chart No. 3D, JHS color chart No. 2904).

Reproductive organs.—1 normal pistil and 5 normal stamens.

Fertility.—Fertile.

Peduncle.—Thin, and 2.7 cm in length.

Physiological and ecological characteristics.—High resistance to rain, cold, rain, heat, drought and diseases such as Powdery Mildew. The resistance to cold, heat, disease and pests are moderate.

This new variety of Petunia plant ‘SUNBEL-APU’ was asexually reproduced by cutting at Youkaichi-shi, Shigaken, Japan, and the homogeneity and stability thereof were confirmed.

### SUMMARY OF THE VARIETY

This new variety of Petunia plant ‘SUNBEL-APU’ has a semi-erect habit with very small pale yellowish pink color with bright yellowish red center margin flowers, and thus is very different from similar varieties ‘SUNBELKI’ and ‘Pearl Sky Blue’. The plant has semi-erect habit, a compact plant shape and abundant branching. The secondary branching is especially strong. The plant has a great profusion of blooms, with the whole plant remaining in bloom for a considerable period of time. The Blooming period is late May to September, and flowers are still open in the rainy season and the flowering duration is long. A whole bush remains in bloom for a considerable period of time. The flowers are very small and the petals have a light yellowish pink coloration with vivid reddish orange margin, which is clearly distinguished from the brilliant yellow petals of ‘SUNBELKI’ and the strong purple petals of ‘Pearl Sky Blue’.

The flower size is very small. The plant is highly tolerant to cold and heat, has a high resistance to pests and diseases, particularly powdery mildew and a high resistance to rain.

### BRIEF DESCRIPTION OF THE DRAWINGS

FIG. 1 is a photograph of flowers of the new variety of petunia plant.

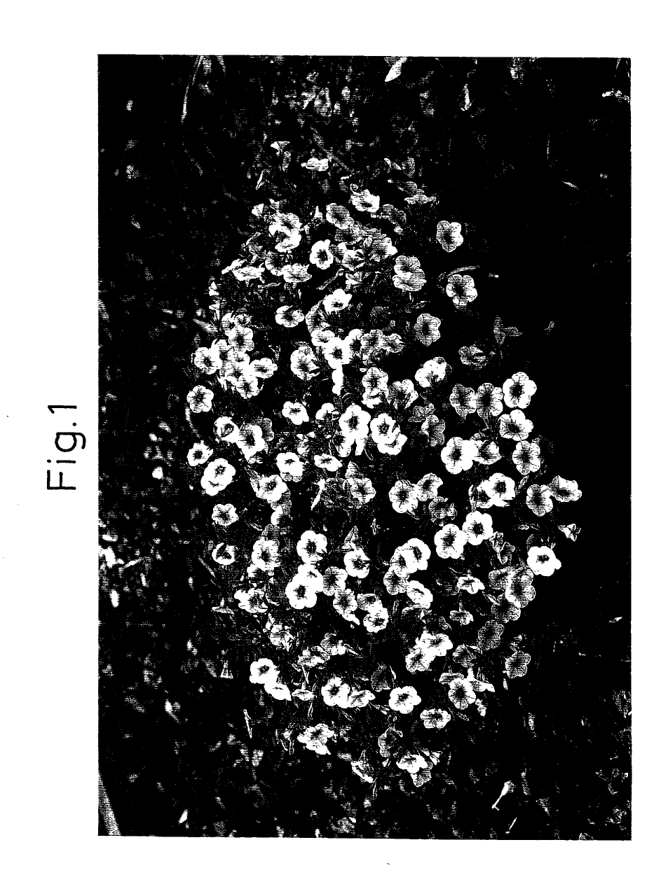

### DESCRIPTION OF THE NEW VARIETY

The botanical characteristics of the new and distinct variety of Petunia plant named ‘SUNBEL-APU’ are as follows, from observation, during November at Youkaichi-shi, Shiga-ken, Japan, of young plants that had been reproduced by cutting during September the year before.

Plant:

Growth habit.—Erect to medium.

Plant height.—Medium; 15 cm.

Spreading area of plant.—The stem extends to a length of 14 cm from the base.

Growth.—Very vigorous with abundant branching, and a great profusion of blooms, with the whole bush remaining in bloom for a considerable period of time.

Blooming period.—May to September in the southern Kanto area, Japan.

Stem:

Thickness.—Thin; 2.3 mm.

Pubescence.—Sparse.

Branching.—Abundant with a superior branching propensity. The secondary branches are particularly numerous.

Length of internode.—Short; 1.4 cm.

Leaf:

Shape.—Lanceolate.

Length.—Very Short; 2.4 cm.

Width.—Very Narrow; 0.8 cm.

Color.—Moderate olive green. (R.H.S. color chart No. 138A, JHS color chart No. 3308).

Thickness.—Medium; 0.6 mm.

Pubescence.—Sparse to medium.

Leaf attachment angle to stem.—Slanted upward to horizontal.

Flower:

Facing direction.—Upward to Slanted upward.

Type.—Single.

Shape.—Funnel-shape, with five petals.

Shape of petal tip.—Obtuse.

Waving of petal.—Weak.

Lobation of petal.—Shallow.

Diameter.—Very small; 2.5 mm.

Variegated pattern of corolla.—Margin.

Ground color of bi-colored petal.—Light yellowish pink (R.H.S. color chart No. 36A, JHS color chart No. 0702).

Color of variegated pattern.—Vivid reddish orange (R.H.S. color chart No. 34A, JHS color chart No. 0705).

Bottom color of the corolla throat.—Vivid yellow (R.H.S. color chart No. 15A, JHS color chart No. 2205).

Outside color of corolla tube.—Light yellow (R.H.S. color chart No. 13C, JHS color chart No. 2203).

Reproductive organs.—1 normal pistil and 5 normal stamens.

Fertility.—Fertile, but self-incompatible.

Peduncle.—0.7 mm in thickness, and 1.2 cm in length.

Physiological and ecological characteristics.—High resistance to cold, rain, heat, drought and diseases such as Powdery Mildew. The resistance to rain and heat is very strong.

Blooming.—A bloom commonly lasts approximately 10 days on the plant. Pinching is not necessary to ensure continued blooming; however, it does tend to enhance bloom production somewhat.

This new variety of Petunia plant is most suitable for flower bedding and potting, particularly in hanging pots and planters, and is further excellent for ground cover usage. The plant of this new variety, ‘SUNBEL-APU’ is presently planted and maintained at Youkaichi-shi, Shiga-ken, Japan.

1. A new and distinct variety of Petunia plant, substantially as herein illustrated and described, characterized particularly as to novelty by (A) having a semi-erect habit, (B) a plentiful number of flowers in a spike and a great profusion blooms, with the entire plant remaining in bloom for a considerable period of time, (C) a long flowering duration, (D) the petals having a light yellowish pink color with vivid reddish orange center margin, (E) a high resistance to rain, cold, heat, drought, diseases and pests, and (F) a late flowering time.
